(19) United States
(12) Patent Application Publication
(10) Pub. No.: US 2012/0109117 A1
(43) Pub. Date: May 3, 2012

(54) CRYOGENIC PROBE WITH SWIVEL

(76) Inventors: Adam Harp, Cincinnati, OH (US); William J. Abner, Cincinnati, OH (US); Jeffrey Stone, Lebanon, OH (US); Salvatore Privitera, Mason, OH (US)

(21) Appl. No.: 12/914,381

(22) Filed: Oct. 28, 2010

(51) Int. Cl. A61B 18/02

(52) U.S. Cl. 606/21; 606/22

(57) ABSTRACT

A cryogenic wand for ablating tissue comprising a cryogenic ablation tube closed at a distal end and including an ablation zone, the ablation tube in fluid communication with and coupled proximally to at least one of two segments that are rotatably repositionable with respect to one another, wherein the two segments are fluidically sealed so that fluid entering a first of the at least two segments passes therethrough and enters a second of the at least two segments.

### RELATED ART

### Field of the Invention

The present disclosure is directed to cryogenic probes and, more specifically, to cryogenic probes that are rotationally repositionable.

### INTRODUCTION TO THE INVENTION

It is a first aspect of the present invention to provide a cryogenic wand for ablating tissue comprising a cryogenic ablation tube closed at one end, the ablation tube in fluid communication with at least two segments that are rotatably repositionable with respect to one another, wherein the two segments are fluidically sealed so that fluid entering a first of the at least two segments passes therethrough and enters a second of the at least two segments.

In a more detailed embodiment of the first aspect, a swivel interposes the at least two segments. In yet another more detailed embodiment, the swivel circumscribes a stationary cryogenic feed conduit supplying fresh cryogenic fluid to the cryogenic ablation tube. In a further detailed embodiment, the cryogenic ablation tube includes a thermocouple. In still a further detailed embodiment, the swivel comprises: (a) a first swivel section defining a first longitudinal cavity extending therethrough, (b) a second swivel section defining a second longitudinal cavity extending therethrough and, (c) a first seal circumscribing at least a portion of the first swivel section and inscribing at least a portion of the second swivel section to form a seal between the first and second swivel sections. In more detailed embodiment, the first longitudinal cavity is in series with the second longitudinal cavity. In a more detailed embodiment, an interior surface defining the first longitudinal cavity also defines a portion of a cryogenic fluid exhaust conduit, an interior surface defining the second longitudinal cavity also defines a portion of the cryogenic fluid exhaust conduit and, the portions of the cryogenic fluid exhaust conduit defined by the first and second longitudinal cavities circumscribe a portion of a cryogenic feed conduit. In another more detailed embodiment, the first longitudinal cavity is coaxial with the second longitudinal cavity, and the first and second longitudinal cavities are at least partially occupied by a cryogenic feed conduit. In yet another more detailed embodiment, the swivel comprises: (a) a first swivel section defining a first longitudinal cavity extending therethrough, (b) a second swivel section defining a second longitudinal cavity extending therethrough, (c) a sleeve concurrently circumscribing at least a portion of the first swivel section and a portion of the second swivel section, and (d) a seal interposing the sleeve and at least one of the first swivel section and the second swivel section. In still another more detailed embodiment, the first swivel section includes a first circumferential recess, the second swivel section includes a second circumferential recess, the sleeve is cylindrical and concurrently circumscribes the first circumferential recess and the second circumferential recess, the seal includes a first O-ring seated within the first circumferential recess to interpose the sleeve and the first swivel section to seal therebetween, and the seal includes a second O-ring seated within the second circumferential recess to interpose the sleeve and the first swivel section to seal therebetween.

In yet another more detailed embodiment of the first aspect, the first swivel section includes a first cylindrical portion, the second swivel section includes a second cylindrical portion, the sleeve houses both the first and second cylindrical portions, and an insulated housing circumscribes the sleeve, the first cylindrical portion, and the second cylindrical portion. In still another more detailed embodiment, the wand further comprises a diverter in fluid communication with the cryogenic ablation tube, the diverter including: (a) a first fitting accommodating an outgoing cryogenic feed conduit and an incoming cryogenic exhaust conduit, (b) a second fitting accommodating an incoming cryogenic feed conduit, and (c) a third fitting accommodating an outgoing cryogenic exhaust conduit, where the second fitting is sealed off from the third fitting. In a further detailed embodiment, the first fitting is operatively coupled to at least one of the at least two segments that are rotatably repositionable with respect to one another. In still a further detailed embodiment, the diverter is positioned within an insulated housing, where the insulating housing includes a first orifice receiving at least two coaxial conduits, where a first of the at least two coaxial conduits is an outgoing cryogenic feed conduit, and where a second of the at least two coaxial conduits is incoming cryogenic exhaust conduit. In a more detailed embodiment, the insulating housing includes a second orifice receiving at least one of an incoming cryogenic feed conduit and an outgoing cryogenic exhaust conduit. In a more detailed embodiment, an incoming cryogenic feed conduit enters the insulating housing and an outgoing cryogenic exhaust conduit leaves the insulating housing, and the incoming cryogenic feed conduit and the outgoing cryogenic exhaust conduit are not coaxially oriented with respect to one another. In another more detailed embodiment, the wand further comprises a robotic appendage coupled to the cryogenic ablation tube in order to facilitate grasping by robotic jaws. In yet another more detailed embodiment, the robotic appendage includes a collar that circumscribes cryogenic ablation tube.

In a more detailed embodiment of the first aspect, the collar defines a longitudinal cavity occupied by at least a portion of the cryogenic ablation tube and an adapter to couple the cryogenic tube to at least one of the at least two segments that are rotatably repositionable with respect to one another. In yet another more detailed embodiment, the wand further comprises an integrated diverter and swivel assembly that interposes the at least two segments that are rotatably repositionable with respect to one another, wherein a first of the at least two segments includes at least two separate conduits for carrying cryogenic feed fluid and exhausted cryogenic fluid, wherein a second of the at least two segments includes at least two separate conduits for carrying the cryogenic feed fluid and the exhausted cryogenic fluid. In a further detailed embodiment, the first of the at least two segments comprises a first swivel section, the second of the at least two segments comprises a second swivel section, the integrated diverter and swivel assembly includes a first seal circumscribing at least a portion of the first swivel section and inscribing at least a portion of the second swivel section to form a seal between the first and second swivel sections. In still a further detailed embodiment, the at least two separate conduits for carrying cryogenic feed fluid and exhausted cryogenic fluid of the first segment are coaxially oriented with respect to one another. In a more detailed embodiment, the at least two separate conduits for carrying cryogenic feed fluid and exhausted cryogenic fluid of the second segment are not coaxially oriented with respect to one another. In a more detailed embodiment, the first of the at least two segments comprises a first swivel section, the second of the at least two segments comprises a second swivel section, the integrated diverter and swivel assembly includes a sleeve concurrently circumscribing at least a portion of the first swivel section and a portion of the second swivel section, the integrated diverter and swivel assembly includes a seal interposing the sleeve and at least one of the first swivel section and the second swivel section. In another more detailed embodiment, the at least two separate conduits for carrying cryogenic feed fluid and exhausted cryogenic fluid of the first segment are coaxially oriented with respect to one another. In yet another more detailed embodiment, the at least two separate conduits for carrying cryogenic feed fluid and exhausted cryogenic fluid of the second segment are not coaxially oriented with respect to one another. In still another more detailed embodiment, the first swivel section includes a first circumferential recess, the second swivel section includes a second circumferential recess, the sleeve is cylindrical and concurrently circumscribes the first circumferential recess and the second circumferential recess, the seal includes a first O-ring seated within the first circumferential recess to interpose the sleeve and the first swivel section to seal therebetween, and the seal includes a second O-ring seated within the second circumferential recess to interpose the sleeve and the first swivel section to seal therebetween. In yet another more detailed embodiment, the first swivel section includes a first cylindrical portion, the second swivel section includes a second cylindrical portion, the sleeve houses both the first and second cylindrical portions, an insulated housing circumscribes the sleeve, the first cylindrical portion, and the second cylindrical portion.

It is a second aspect of the present invention to provide a cryogenic probe for ablating tissue comprising: (a) an elongated tube open at one end and closed at an opposite end, the elongated tube operatively coupled to at least two segments that are rotatably repositionable with respect to one another using an in-series swivel, wherein the two segments are sealed to inhibit fluid passing between the at least two segments, the elongated tube defining an internal cavity; and, (b) at least one cryogenic fluid supply line occupying at least a portion of the internal cavity of the elongated tube, the at least one cryogenic fluid supply line including a nozzle for introducing cryogenic fluid into the internal cavity of the elongated shell.

In a more detailed embodiment of the second aspect, the at least one cryogenic fluid supply line includes at least two sections that are rotatably repositionable with respect to one another. In yet another more detailed embodiment, the elongated tube includes a wall thickness of from about 0.020 inches to 0.035 inches. In a further detailed embodiment, the elongated tube includes an outside diameter of from about 0.16 inches to about 0.20 inches. In still a further detailed embodiment, the cryogenic probe further comprises a swivel interposing the at least two segments. In a more detailed embodiment, the cryogenic probe further comprises a diverter in fluid communication with the elongated tube, the diverter including: (a) a first fitting accommodating an outgoing cryogenic feed conduit and an incoming cryogenic exhaust conduit, (b) a second fitting accommodating an incoming cryogenic feed conduit, and (c) a third fitting accommodating an outgoing cryogenic exhaust conduit, where the second fitting is sealed off from the third fitting. In a more detailed embodiment, the cryogenic probe further comprises a thermocouple mounted to the elongated tube. In another more detailed embodiment, the cryogenic probe further comprises a metallic coil housed within the elongated tube.

It is a third aspect of the present invention to provide a cryogenic probe for ablating tissue comprising: (a) an ablator comprising an ablation tube closed at one end and occupied by a cryogenic feed tube having an orifice, where an interior surface of the ablator at least partially defines a cryogenic exhaust conduit, where the ablation tube includes a wall thickness of from about 0.020 inches to 0.035 inches, and where the ablation tube includes an outside diameter of from about 0.16 inches to about 0.20 inches; and, (b) a swivel operatively coupled to the ablator to allow the ablation tube to rotate about the cryogenic feed tube.

It is a fourth aspect of the present invention to provide a method of fabricating a cryogenic wand for ablating tissue, the method comprising: (a) forming a cryogenic ablation tube closed at one end; (b) operatively coupling the cryogenic ablation tube to a swivel, where the swivel interposes the closed end of the cryogenic ablation tube and a conduit adapted to carry at least one feed cryogenic fluid proximate the closed end of the cryogenic ablation tube and exhaust cryogenic fluid returning from proximate the closed end of the cryogenic ablation tube.

In a more detailed embodiment of the fourth aspect, the method further includes the act of inserting a cryogenic feed conduit within the swivel so the swivel circumscribes the cryogenic feed conduit. In yet another more detailed embodiment, the method further includes the act of inserting mounting a thermocouple to the cryogenic ablation tube. In a further detailed embodiment, the swivel comprises: (a) a first swivel section defining a first longitudinal cavity extending therethrough; (b) a second swivel section defining a second longitudinal cavity extending therethrough; and, (c) a first seal circumscribing at least a portion of the first swivel section and inscribing at least a portion of the second swivel section to form a seal between the first and second swivel sections. In still a further detailed embodiment, the first longitudinal cavity is in series with the second longitudinal cavity. In a more detailed embodiment, an interior surface defining the first longitudinal cavity also defines a portion of a cryogenic fluid exhaust conduit, an interior surface defining the second longitudinal cavity also defines a portion of the cryogenic fluid exhaust conduit, and the portions of the cryogenic fluid exhaust conduit defined by the first and second longitudinal cavities circumscribe a portion of a cryogenic feed conduit. In a more detailed embodiment, the first longitudinal cavity is coaxial with the second longitudinal cavity, and the first and second longitudinal cavities are at least partially occupied by a cryogenic feed conduit. In yet another more detailed embodiment, the swivel comprises: (a) a first swivel section defining a first longitudinal cavity extending therethrough; (b) a second swivel section defining a second longitudinal cavity extending therethrough; a sleeve concurrently circumscribing at least a portion of the first swivel section and a portion of the second swivel section; and, (d) a seal interposing the sleeve and at least one of the first swivel section and the second swivel section. In still another more detailed embodiment, the first swivel section includes a first circumferential recess, the second swivel section includes a second circumferential recess, the sleeve is cylindrical and concurrently circumscribes the first circumferential recess and the second circumferential recess, the seal includes a first O-ring seated within the first circumferential recess to interpose the sleeve and the first swivel section to seal therebetween, and the seal includes a second O-ring seated within the second circumferential recess to interpose the sleeve and the first swivel section to seal therebetween.

In yet another more detailed embodiment of the fourth aspect, the first swivel section includes a first cylindrical portion, the second swivel section includes a second cylindrical portion, the sleeve houses both the first and second cylindrical portions, an insulated housing circumscribes the sleeve, the first cylindrical portion, and the second cylindrical portion. In still another more detailed embodiment, the method further includes the act of operatively coupling a diverter to the swivel, the diverter including: (a) a first fitting accommodating an outgoing cryogenic feed conduit and an incoming cryogenic exhaust conduit, (b) a second fitting accommodating an incoming cryogenic feed conduit, and (c) a third fitting accommodating an outgoing cryogenic exhaust conduit, where the second fitting is sealed off from the third fitting. In a further detailed embodiment, the first fitting is operatively coupled to at least one of the at least two segments that are rotatably repositionable with respect to one another. In still a further detailed embodiment, the diverter is positioned within an insulated housing, where the insulating housing includes a first orifice receiving at least two coaxial conduits, where a first of the at least two coaxial conduits is an outgoing cryogenic feed conduit, and where a second of the at least two coaxial conduits is incoming cryogenic exhaust conduit. In a more detailed embodiment, the insulating housing includes a second orifice receiving at least one of an incoming cryogenic feed conduit and an outgoing cryogenic exhaust conduit. In a more detailed embodiment, an incoming cryogenic feed conduit enters the insulating housing and an outgoing cryogenic exhaust conduit leaves the insulating housing, and the incoming cryogenic feed conduit and the outgoing cryogenic exhaust conduit are not coaxially oriented with respect to one another.

In yet another more detailed embodiment of the fourth aspect, the method further comprises the act of mounting a robotic appendage to the cryogenic ablation tube in order to facilitate grasping by robotic jaws. In still another more detailed embodiment, the robotic appendage includes a collar that circumscribes cryogenic ablation tube. In a further detailed embodiment, the collar defines a longitudinal cavity occupied by at least a portion of the cryogenic ablation tube and an adapter to couple the cryogenic tube to at least one of the at least two segments that are rotatably repositionable with respect to one another. In still a further detailed embodiment, the swivel comprises an integrated diverter and swivel assembly, wherein the integrated diverter and swivel assembly is operative to take an input of two coaxial conduits and create an output of two non-coaxial conduits.

### BRIEF DESCRIPTION OF THE DRAWINGS

FIG. 1 is a schematic diagram of a first exemplary cryogenic probe in accordance with the instant disclosure.

FIG. 2 is a cross-sectional view of the exemplary diversion section of FIG. 1.

FIG. 3 is a cross-sectional view of the exemplary swivel section of FIG. 1.

FIG. 4 is a cross-sectional view of the exemplary tool attachment section of FIG. 1.

FIG. 5 is a schematic diagram of a second exemplary cryogenic probe in accordance with the instant disclosure.

FIG. 6 is a cross-sectional view of the exemplary swivel section of FIG. 5.

FIG. 7 is a cross-sectional view of the exemplary diversion section of FIG. 5.

FIG. 8 is a schematic diagram of a third exemplary cryogenic probe in accordance with the instant disclosure.

FIG. 9 is a cross-sectional view of the exemplary integrated swivel diverter assembly of FIG. 8.

FIG. 10 is a magnified view of the portion identified 9-9 in FIG. 9.

### DETAILED DESCRIPTION

The exemplary embodiments of the present disclosure are described and illustrated below to encompass cryogenic surgical instruments and, more particularly, to cryogenic probes or cryoprobes incorporating at least one swivel and used for creating lines of ablation on tissue such as, for example, for the treatment of cardiac arrythmias including atrial fibrillation. Of course, it will be apparent to those of ordinary skill in the art that the embodiments discussed below are exemplary in nature and may be reconfigured without departing from the scope and spirit of the present disclosure. However, for clarity and precision, the exemplary embodiments as discussed below may include optional steps, methods, and features that one of ordinary skill should recognize as not being a requisite to fall within the scope of the present disclosure. Hereinafter, the exemplary embodiments of the present disclosure will be described with reference to the drawings.

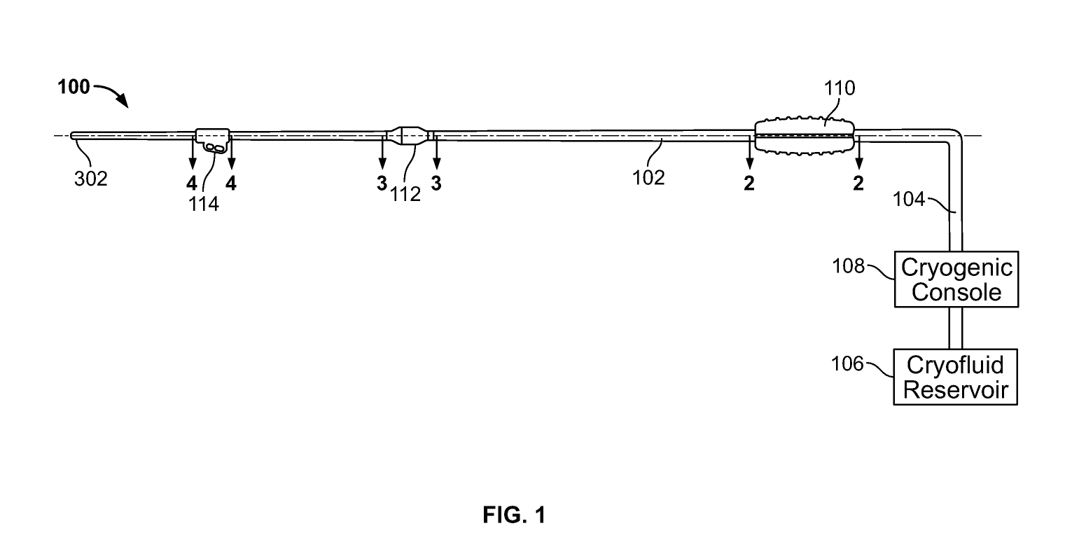

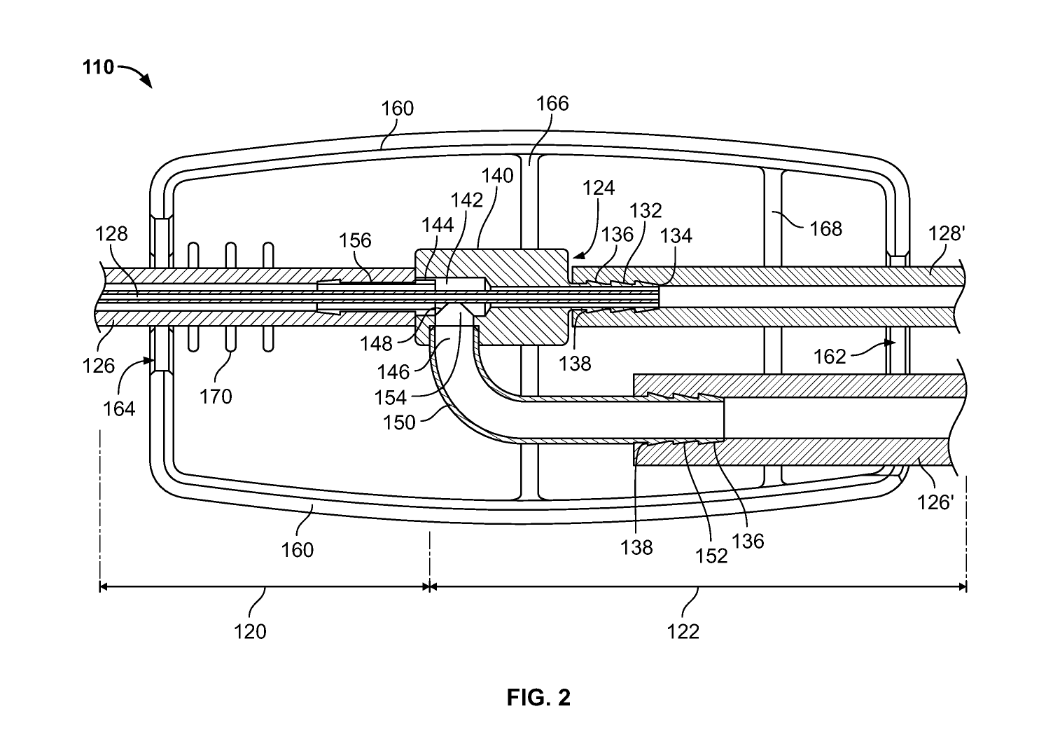

With reference to FIGS. 1 and 2, a first exemplary cryogenic probe 100 includes a wand 102 coupled to an umbilical tether 104 in order to supply cryogenic fluid from a fluid reservoir 106 to the wand. In this first exemplary embodiment, the flow of cryofluid to and from the cryogenic probe 100 is controlled from a separate, commercially available console 108 that regulates and controls the pressure of the cryofluid introduced into the cryogenic wand 102. The console 108 is capable of pressurizing the wand 102 for active defrost and provides for appropriate discharge of expanded cryofluid exhaust. Alternatively, or in addition, hand controls (not shown) may be associated with the cryogenic probe 100 to regulate and control the pressure of the cryofluid introduced into the cryogenic wand 102. In this first exemplary embodiment, the wand 102 includes a diversion section 110, a swivel section 112, and a tool attachment section 114 along the length thereof.

Referring to FIGS. 1 and 2, the diversion section 110 operates to delineate between the coaxial section 120 and the parallel section 122 by housing a diverter 124 operative to provide coaxial flow of the feed and exhaust cryogenic fluid on one side and non-coaxial flow of the exhaust and feed cryogenic fluid on the opposite side. As used herein, the term “diverter” means an apparatus operative to take at least two non-circumscribing conduits and redirects at least one of the conduits so that a first of the conduits at least partially circumscribes at least a second of the conduits. In the parallel section 122, the exhausted cryogenic fluid is carried away from the ablation end (i.e., distal end) by a separate exhaust conduit 126′, while incoming cryogenic fluid (approximate a proximal end) is carried toward the ablation end by a feed line 128′ that is not coaxial with the exhaust conduit 126. But on the opposite side of the diverter 124, a feed conduit 128 carrying cryogenic fluid and the exhaust conduit 126 are coaxial, with the exhaust cryogenic fluid flowing over the outside of the feed conduit.

The diverter 124 comprises a cryogenic feed barb fitting 132 that includes a smooth cylindrical cavity sized to receive the cryogenic feed conduit 128 and create a friction fit therewith. In this exemplary embodiment, the proximal end 134 of the barb fitting 132 marks the origination point of the cryogenic feed conduit 128, while the feed line 128′ in communication by the cryogenic fluid tank extends distally past the proximal end to circumscribe the barb fitting 132. The barb fitting 132 includes three barbs characterized by an inclined face (from proximal to distal) and a vertical wall intersecting the inclined face. The inclined faces 136 provide a conical profile that allows the feed line to more easily circumscribe the fitting 132 and be repositioned distally, while the vertical walls 138 retard proximal movement of the feed line after the feed line is installed around the fitting. The barb fitting 132 is an integral part of a diverter body 140 having a longitudinal cavity through which the feed conduit 128 extends. The profile of the longitudinal cavity 142 changes from proximal to distal, with the distal portion being sized only to accommodate the cryogenic feed conduit 128, but moving distally, the profile increases.

The diverter body 140 includes a T-fitting 144 that operates to change the profile of the longitudinal cavity 142. The base section 146 of the T-fitting 144, which extends perpendicularly with respect an adapter section 148 and is in fluid communication therewith, is circumscribed by a branch conduit 150 having its own barb fitting 152 at the proximal end thereof. This barb fitting 152 is similar to the barb fitting 132 discussed above in terms of having inclined faces to provide a conical profile that allows the separate exhaust conduit 126′ to more easily circumscribe the fitting 152 and vertical walls to retard proximal movement of the separate exhaust conduit after the separate exhaust conduit is installed around the fitting 152. In exemplary form, the branch conduit 150 is at least one of welded, brazed, and soldered to the base section 146 to ensure a fluid tight seal therebetween.

In order to divert the exhaust cryogenic fluid through the branch conduit 150, the adapter section 148 is sealed at its proximal end and is large enough to provide a circumferential gap 154 between the interior of the branch conduit and the exterior of the feed conduit 128. This circumferential gap provides a pathway for exhaust cryogenic fluid passing from the exhaust conduit 126 to reach the base section 146, flow into the branch conduit 150, and ultimately flow into the separate exhaust conduit 126′.

In order to mount the exhaust conduit 126 to the diverter body 140, the diverter body includes a distal barb fitting 156 extending from, and in fluid communication with, the adapter section 148. The barb fitting 156 is received within the distal portion of the adapter section 148 and create a friction fit therebetween to retain the fitting. However, in this exemplary embodiment, the fitting is at least one of welded, brazed, and soldered to the adapter section 148 to ensure a fluid tight seal therebetween. The distal portion of the barb fitting 156 includes at least one barb, similar to those discussed above, in order to be circumscribed by the exhaust conduit 126 and retard distal movement of the exhaust conduit away from the barb fitting. Though not necessarily required, the exhaust conduit 126 may be at least one of welded, brazed, and soldered to the barb fitting 156 to ensure a fluid tight seal therebetween.

The diverter 124 is seated within a housing 160 comprised of complementary halves. It is to be understood, however, that the housing may be a single piece, such as an injection molded jacket. The housing 160 includes two openings 162, 164 oriented at opposite ends of the housing, with the larger of the two openings 162 located at a proximal end of the housing and allowing throughput of both the separate exhaust conduit 126′ and separate feed conduit 128′, whereas the smaller of the two openings 164 at the distal end of the housing allows throughput of the coaxial conduits 126, 128. The interior of the housing 160 includes a series of ribs 166, 168, 170 that protrude from an interior surface and operate to align the diverter 124 and conduits 126, 128, 126′, 128′. Distal from the diversion section 110 is the swivel section 112.

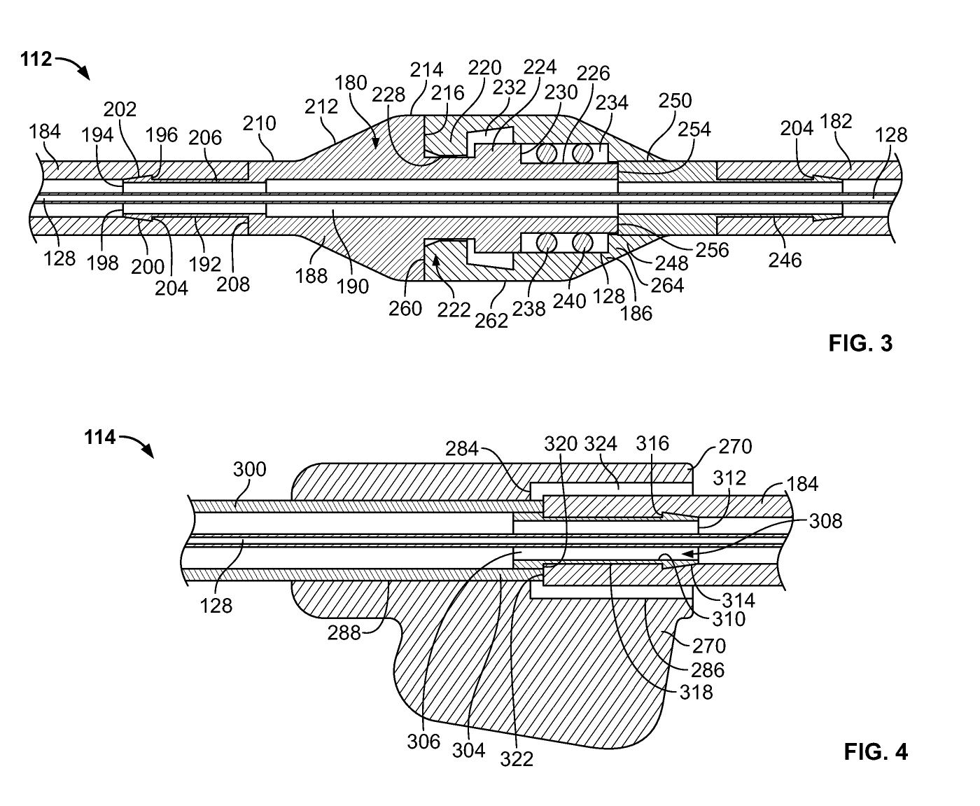

Referring to FIGS. 1 and 3, the swivel section 112 includes a swivel 180 so that the proximal and distal sections 182, 184 of the exhaust conduit 128 can rotate with respect to one another. In this exemplary embodiment, the swivel 180 includes a proximal component 186 and a distal component 188 that are rotationally repositionable with respect to one another. Each component 186, 188 includes a longitudinal passage 190 defined by a series of interior surfaces of the components that accommodates throughput of the feed conduit 128, but is also large enough to accommodate a separate stream of exhaust cryogenic fluid. More specifically, the interior circumferential surface of the feed conduit 128 provides a sealed delivery mechanism for the cryogenic fluid delivered from the cryogenic fluid tank. At the same time; the interior surfaces of the components 186, 188 cooperate with the exterior surface of the feed conduit 128 to define a sealed delivery mechanism for the exhausted cryogenic fluid.

The distal component 188 includes an integrally formed barb fitting 192 having a circular opening 194 that leads into a cylindrical cavity bounded by an interior surface 196 having a substantially constant diameter. The circular opening 194 is defined by a circular lip 198 that transitions into the exterior surface 200 of the barb fitting 192. This exterior surface 200 is characterized by a circular cross-section that, unlike the interior surface 196, changes to taper outward from the lip 198 to increase its diameter. This tapered section 202 increases in diameter until terminating at a vertical step 204 to form the barb. This vertical step 204 operates to retard the distal section 184 of the exhaust conduit from substantial distal movement that would remove the conduit from the barb fitting 192. Beyond the barb, traveling proximally, the exterior surface includes a substantially cylindrical, constant diameter section 206 that extends to reach a second vertical step 208. This second vertical step 208 is adjacent to a constant diameter section 210 that is adjacent to a conical section 212 having a diameter that increases (distal to proximal), which is itself adjacent to another constant diameter section 214 marking the maximum widthwise dimension of the swivel 180. Immediately proximal to this constant diameter section 214 is a vertical wall 216 that marks the transition for the portion of the exterior surface 200 of the distal component 188 that interfaces with the interior surfaces 128 of the proximal component 186.

In order to resist longitudinal motion of the components 186, 188 away from one another, the proximal component 186 includes a circumferential end flange 220 that is received within a circumferential groove 222 of the distal component. This circumferential flange 220 is tapered on its distal end (i.e., leading edge) in order to pass over a plateau 224 that extends circumferentially beyond an external barrel surface 226. One side of the plateau 224 is joined to the vertical wall 216 by a recessed wall 228 that all cooperate to define the groove 222. In order to retain the circumferential end flange 220 within the circumferential groove 222, the backside 230 of the flange is vertical so that the backside is parallel to the front side of the plateau 224 defining the groove. The interior surface 128 of the proximal component 186 partially defines a pair of circumferential cavities 232, 234 in which at least one seal ring is located.

In this exemplary embodiment, the first circumferential cavity 232 has a trapezoidal cross-section, but is unoccupied by a seal ring. However, in an alternate exemplary embodiment, the cavity 232 may be occupied by one or more seal rings. The second circumferential cavity 234 has a rectangular cross-section cooperatively defined by the backside of the plateau 224, the barrel surface 226, and the interior surface 128 extending in parallel to the barrel surface. In exemplary form, the second circumferential cavity 234 is at least partially occupied by a pair of sealing rings 238, 240. The sealing rings 238, 240 allow the components 186, 188 to rotate with respect to one another while a fluid tight seal is maintained therebetween. In this exemplary embodiment, the seal rings 238, 240 have different cross-sectional shapes, with the first seal ring 238 (nearest to the plateau 224) having a rectangular cross-section, while the second seal ring 240 (nearest to the proximal tip) includes a circular cross-section.

Those skilled in the art will realize that fewer or more than two seal rings may be utilized. Moreover, if multiple seal rings are utilized, any of various shapes of seal rings may be utilized. In addition, if multiple seal rings are utilized, the seal rings may all have the same cross-sectional shape and size, or the seal rings may have different shapes and/or sizes.

Referring back to FIGS. 1 and 3, the proximal component 186 also includes an integrally formed barb fitting 246 having generally the same shape and features as the barb fitting 192, of the distal component 188, except in the opposite direction. Accordingly, like elements of the barb fittings 192, 246 have been labeled commonly in the figures. Unlike the barb fitting 192 of the distal component 188, the barb fitting 246 of the proximal component 186 is not integrally formed as a single piece with the housing 248 of the proximal component. Instead, the barb fitting 246 is a separate structure that is at least one of welded, brazed, and soldered to join the fitting with the housing 248 to ensure a fluid tight seal at the joint 250. In exemplary form, the housing 248 includes a proximal opening that extends into a cylindrical cavity partially occupied by the barb fitting and the distal component 188.

The dimensions of the cylindrical cavity generally match those of the exterior of the barb fitting 246 in order to retain the barb fitting therein by way of a compression fit. The cavity is also partially defined by a step 254 providing a depthwise endpoint upon which a portion of the base 256 of the barb fitting 246 is seated. This step 254 also marks the proximal endpoint of the distal component 188. In exemplary form, the proximal end of the distal component 188 abuts the base 256 of the barb fitting 246 when the vertical wall 216 abuts the distal end 260 of the proximal component 186. However, because of the distance between the front side of the plateau 224 the vertical wall 216 is greater than the width (i.e., depth) of the circumferential end flange 220, there is the potential for longitudinal play between the components 186, 188. But this play does not substantially affect the outer shape of the housing 248 which includes a cylindrical section 262 and a tapering section 264 that decreases in diameter from distal to proximal until terminating at the exterior of the barb fitting 246. In this manner, the swivel 180 is tapered on both sides to reduce the likelihood of snagging when moved longitudinally forward or rearward. As with the distal barb fitting 192, the proximal barb fitting 246 includes a vertical step 204 operative to retard the proximal section 182 of the exhaust conduit from substantial proximal movement that would remove the conduit from the barb fitting 246. Distal from the swivel section 112 is the tool attachment section 114.

Referring to FIGS. 1 and 4, the tool attachment section 114 includes a collar 270 and a radially extending tab 272. This tab 272 includes a top and opposed bottom surfaces 276, 278 each having at least one projection 280 extending either or both surfaces. In this exemplary embodiment, each surface 276, 278 of the tab 272 includes a pair of perpendicularly extending, oblong projections 280. These projections 280 help a set of robotic jaws (now shown) grasp the tab 272 and correspondingly reposition the wand 102.

In exemplary form, the collar 270 is fabricated from any suitable, biocompatible material and includes a through passage that receives the distal section 184 of the exhaust conduit 128 attached to the swivel section 112. The through passage has a circular cross-section that is non-uniform. A vertical step 284 interposes an inner circumferential wall 286 and another inner circumferential wall 288. The vertical step 284 is operative to decrease the diameter of the passage from the larger diameter walls 286 to the smaller diameter walls 288. Both sets of walls 284, 288 have a substantially constant (but different) diameter to define cylindrical cavities that are longitudinally aligned and sequential with respect to one another.

One section 184 of the exhaust conduit is located within the proximal cavity, while a boiler tube 300 is frictionally mounted to the collar 270 within the distal cavity using a friction fit. In exemplary form, the boiler tube 300 is hollow and includes a closed distal end 302 and an open proximal end 304. This open proximal end 304 is sized to receive a barb fitting 306. The barb fitting 306 is a separate structure that is at least one of welded, brazed, and soldered to the interior of the boiler tube 300 to ensure a fluid tight seal therebetween.

Consistent with the structure of the foregoing barb fittings, this barb fitting 306 also includes a circular opening 308 that leads into a cylindrical cavity bounded by an interior surface 310 having a substantially constant diameter. The circular opening 308 is defined by a circular lip 312 that transitions into the exterior surface 314. This exterior surface 314 tapers outward from the lip 312 to increase its diameter, terminating at a vertical step 316 to form the barb. This vertical step 316 operates to retard the distal section 184 of the exhaust conduit from substantial distal movement that would remove the conduit from the barb fitting 306. Beyond the barb, traveling proximally, the exterior surface includes a substantially cylindrical, constant diameter section 318 that extends to reach a second vertical step 320. This second vertical step 320 is coplanar with the open 322 of the boiler tube 300 and signals an increase in the thickness of the fitting 306 wall to approximate the internal diameter of the boiler tube 300.

The end 322 of the boiler tube 300 extends partially into the proximal cavity. This cavity is sized to receive the boiler tube 300, the barb fitting 306, as well as a portion of the distal section 184 of the exhaust conduit. Specifically, the diameter of the proximal cavity is greater than the outside diameter of the distal section of the exhaust conduct, which thus leaves a circumferential gap 324.

The boiler tube 300 includes a generally circular cross section and a longitudinal cylindrical profile when oriented in a linear fashion. However, the tube 300 may be deformed to take on shapes, other than a linear orientation such as a candy cane shape. In this circumstance, the tube 300 comprises an aluminum shell having a nominal wall thickness ranging between about 0.020 in. to about 0.035 in. and an outside diameter of from about 0.16 in. to about 0.20 in. This wall thickness allows the tube 300 to be deformed in an arcuate manner without causing a crack or other breach allowing communication between the exterior and interior of the shell.

It should be noted that in lieu of aluminum, other materials may be used to construct the tube 300. For example, the tube 300 may be fabricated from Series 1000 aluminum alloy or other metals and alloys including, without limitation, gold, gold alloys, stainless steel, nitinol, or other malleable metals and metallic alloys having suitable thermal conductivity. In exemplary form, the tube 300 is formable into various shapes appropriate for making the different ablation lines, but is stiff enough for tissue conformance and to maintain its shape when applied to cardiac tissue without any secondary reinforcement. Likewise, the exemplary tube 300 is capable of being bent in an arcuate manner to have a minimum radius of approximately 0.5 in.

The tube 300 has a smooth outer surface over the vast majority of its length. In this exemplary embodiment, the tube 300 has an overall length of approximately 43 cm. It should be noted, however, the lengths longer than 43 cm and shorter than 43 cm may also be utilized. In order to house and encase the cryogenic fluid flowing within the tube 300, the interior of the tube 300 is generally hollow and enclosed at the distal end 302. In exemplary form, the distal tip of the tube 300 embodies a generally hemispherical shape.

On the interior of the tube 300 is the feed conduit 128 that directs cryogenic fluid proximate the closed end 302. In this exemplary embodiment, the feed conduit 128 includes a generally circular cross section and a longitudinal cylindrical profile when oriented in a linear fashion. However, the feed conduit 128 may be deformed to take on shapes other than a linear orientation. In this exemplary circumstance, the feed conduit 128 comprises an aluminum shell having a nominal wall thickness ranging between 0.02-0.035 inches and a nominal internal diameter ranging between 0.005-0.012 inches. It should be noted that in lieu of aluminum, other materials may be used to construct the feed conduit 128. For example, the feed conduit 128 may be fabricated from Series 1000 aluminum alloy or other metals and alloys including, without limitation, gold, gold alloys, stainless steel, nitinol, or other malleable metals and metallic alloys having suitable thermal conductivity.

The wall thickness allows the feed conduit 128 to be deformed in an arcuate manner to generally track the deformity in the tube 300. In this exemplary embodiment, the feed conduit 128 includes a nozzle (not shown) having a cross-sectional area that achieves a flow rate of 1,000-1,600 cubic centimeters per minute at 15 psi. The nozzle is backset from the enclosed end 302 of the tube 300. By way of example, and not limitation, the inside diameter of the feed conduit 128 generally has a length from about 10 inches to about 20 inches and a corresponding cross sectional area generally from about 0.000314 square inches to about 0.00196 square inches. It is also within the scope of the disclosure to provide multiple nozzles, some of which may be staggered along the length of the tube 300.

Though not required, the interior of the tube 300 may include an internal flexible support, such as a metal coil (not shown), to prevent kinking and to help maintain the circular cross-section of the tube during deformation. In this exemplary embodiment, the flexible support comprises a stainless steel coil. The flexible support may also serve to hold together segments of the tube 300 in the event that the tube fractures. The flexible support may be free-floating, or it may be retained in place on the interior of the tube 300 by frictional engagement with the inner wall of the shell using oversized coils. In this exemplary embodiment, the spring 332 has a pitch generally ranging between about 0.018 in. to about 0.022 in. and an outside diameter generally ranging between about 0.115 in. to about 0.125 in.

In use, the exterior surface of the tube 300 has surface temperatures below −40° C. When the tube 300 is applied to the tissue to be treated, freezing of tissue coming into direct contact with the shell results. Surrounding tissue is sequentially frozen by the withdrawal of heat from the tissue as the tube 300 maintains contact with tissue over time.

Surfaces of the cryogenic wand 102 that are not intended for potential contact with patient tissue may be insulated for the protection of both non-target tissue and the cryogenic probe user. To this end, a sleeve (not shown) may circumscribe portions of the wand, thus protecting adjacent tissue that may come into contact with what would otherwise be exposed portions of the wand 102.

The cryogenic probe 100 may be used in an open procedure on an arrested heart, with the shell 106 being applied to the endocardium or inner surface of the heart (through a purse-string opening), or alternatively to the epicardium or outer surface of the heart. The freezing of the cardiac tissue causes an inflammatory response (cryonecrosis) that blocks the conduction of electrical pulses.

All materials used in the cryogenic probe 100 that are exposed to the cryofluid may be compatible with the cryofluid used in the device, and components intended for patient contact may be biocompatible. The device (and its packaging) may also be gamma stable, as gamma sterilization is an exemplary sterilization method.

As is known to those skilled in the art, a cryogenic device operates as a result of a cryofluid undergoing expansion as the fluid moves through the nozzle of the feed conduit 128 and into the open cavity of the tube 300. By way of example, the feed conduit 128 may deliver cryogenic fluid at a relatively high pressure, which drastically reduces its pressure after exiting the supply tube and entering the tube proximate its end 302. Temperatures within the interior of the tube 300 can fall below −60° C., and provide for exterior surface temperatures of the shell of less than −45° C., for example, when nitrous oxide gas is used as the cryofluid.

The inventors have found that it may be advantageous to utilize the exhaust cryogenic fluid to reduce the temperature of the feed cryogenic fluid. Accordingly, the first exemplary cryogenic probe 100 makes use of a countercurrent flow pattern within the coaxial section 120 (see FIG. 2) so that the exhaust conduit 126 circumscribes and longitudinally extends around the feed conduit 128. In this manner, the feed conduit leaving the tank includes cryogenic fluid a pressure ranging between 650-900 psi at a temperature of 22 C and ultimately delivers the cryogenic fluid at the nozzle at a pressure of 700 psi and a temperature of −55 C.

While not shown in the figures, it is also within the scope of the invention to provide multiple feed conduits 128. As a result, each of the multiple feed conduits 128 includes a separate passageway for cryogenic fluid to enter the tube 300.

In addition, the cryogenic probe 100 may be provided with a system for determining the surface temperature of the tube 300 and provide the user with that data. To this end, the outer surface of the tube 300 may be provided with a temperature measuring device, such as a thermocouple. Wiring on the outside of the tube 300 transmits signals generated by the thermocouple to a display associated with the cryogenic console 108 having a read-out visible to the user. By way of example, the thermocouple may be a type T calibration thermocouple which is suitable ranging between −250° C. and 350° C.

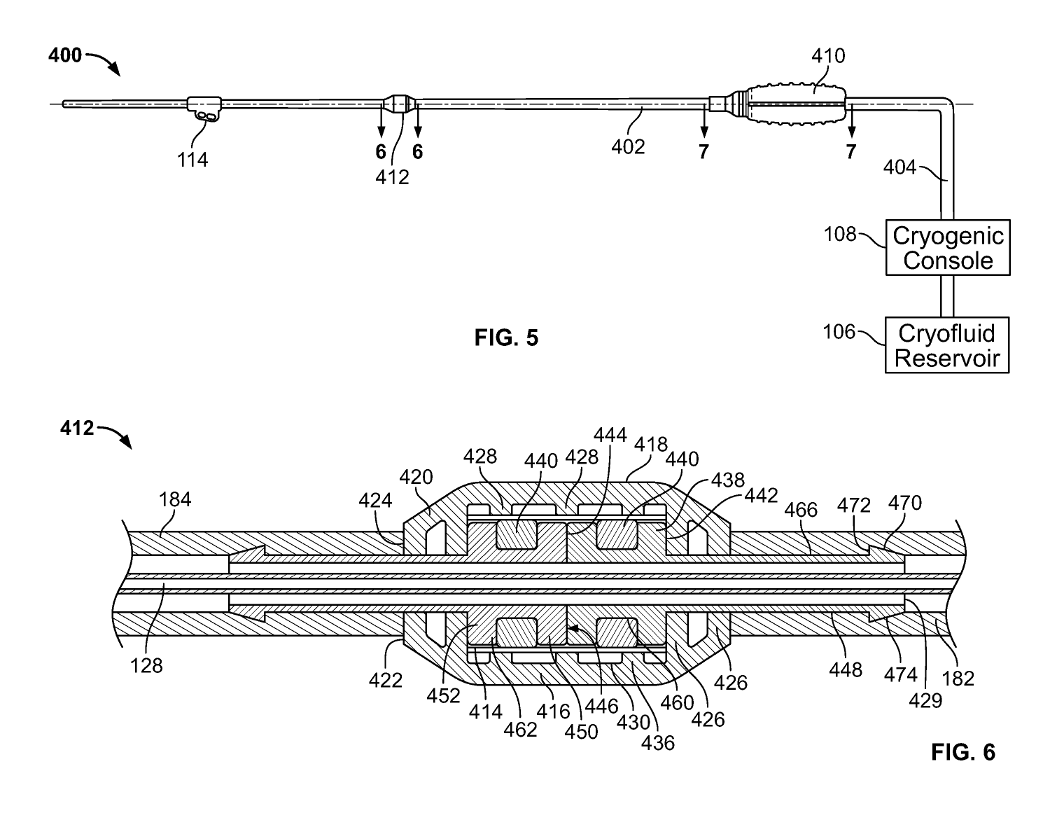

Referring to FIG. 5, a second exemplary cryogenic probe 400 includes a wand 402 coupled to an umbilical tether 404 in order to supply cryogenic fluid from a fluid reservoir 106 to the wand. In this second exemplary embodiment, the flow of cryofluid to and from the cryogenic probe 400 is controlled from a separate, commercially available console 108 that regulates and controls the pressure of the cryofluid introduced into the cryogenic wand 402. The console 108 is capable of pressurizing the wand 402 for active defrost and provides for appropriate discharge of expanded cryofluid exhaust. Alternatively, or in addition, hand controls (not shown) may be associated with the cryogenic probe 400 to regulate and control the pressure of the cryofluid introduced into the cryogenic wand 402. In this second exemplary embodiment, the wand 402 includes a diversion section 410, a swivel section 412, and a tool attachment section 114. Because this second exemplary probe 400 makes use of the same cryogenic fluid reservoir 106, console 108, and tool attachment section 114, a detailed description of these components will not be duplicated for purposes of brevity.

Referring to FIGS. 5 and 6, the swivel section 412 includes a swivel 414 surrounded by an insulated housing 416 fabricated from complementary plastic halves. Each half includes a generally smooth exterior surface having a middle section 418 with a substantially constant radius that transitions into semi-conical sections 420 having a frustum 422. Each frustum 422 includes a semi-circular opening 424 that is longitudinally aligned with the opposing frustum. An interior of each housing 416 half includes two different shaped sets of semi-circular ribs 426, 428 that protrude from an interior housing wall 430 and toward the cryogenic feed line 128. The first set of ribs 426, comprising a corresponding pair on each end, one defining the frustum 422 and another being inset a predetermined distance from the frustum 422 and operate to define a semi-circular opening 434 having generally the same dimensions as the semi-circular opening 424 of the frustum 408. As can be seen in the drawings, the radial length of the first set of ribs 426 is substantially greater than the radial length of the second set of ribs 428. In this exemplary embodiment, the second set of ribs 428 are three in number and are oriented in between the first set of ribs 426. The difference in radial length between the ribs 426, 428 at least partially defines an internal cavity occupied by a scaling sleeve 436, swivel segments 438, and sealing rings 440.

The sealing sleeve 436 comprises a metallic cylinder having hollow interior and opposing open ends. The exterior surface of the sleeve 436 is generally smooth and circular, as is the interior surface. In this exemplary embodiment, the hollow interior defines a cylindrical cavity having a circular cross-section of substantially identical diameter along its length. By way of example, and not limitation, the wall thickness of the sleeve is 1 millimeter. However, it should be understood that the wall thickness may depend upon the material properties of the sleeve. The other dimensions, such as the length, width (diameter), may be dictated by the dimensions of the swivel segments 438 and sealing rings 440. However, it should be apparent to those skilled in the art that the dimensions are a matter of design choice. In this exemplary embodiment, the exterior surface of the sleeve 436 is adjacent to the second set of ribs 442 from each housing 416 half. Opposing open ends of the sleeve 436 abut complementary interior radial surfaces 442 of the first set of ribs 426 in order to wedge the sleeve between the first set of ribs. Upon assembly of the swivel 414, as will be discussed below, the sleeve 436 does not appreciably move radially outward or longitudinally while seated within the housing 416 halves.

The complementary interior radial surfaces 442 of the first set of ribs 426 also operate to retain the swivel segments 438 and sealing rings 440 within the sleeve 436 when the housing 416 halves are assembled. Specifically, each swivel segment 438 includes a base 444 having a through hole 446 that is generally centered within the base. This through hole 446 extends through the base 444 and through an adjoining conduit 448 until terminating at the end 450 of the conduit. In this exemplary embodiment, the through hole 446 is bounded by a continuous interior surface having a generally constant diameter from the base 444 to proximate the end 449 of the conduit 448 where the interior surface tapers outward to gradually increase the diameter until reaching the end of the conduit. The simplicity of the interior topography contrasts the complexity of the exterior topography.

The exterior topography of each swivel segment 438 includes the substantially flat, circular base 444 discussed above. This base 444 extends radially outward from the through hole 446 to create a circumferential flange 450. A second circumferential flange 452 is longitudinally spaced apart from the first flange 450 and cooperates with the first flange to define a trough into which, the scaling ring 440 is seated. By way of example, and not limitation, the trough has a generally block U-shaped cross-section resulting from opposing walls of the flanges 450, 452 intersecting a circular, inset 460 wall. The depth and the width of the trough may be established based upon the desired dimensions of the sealing ring 440 so that the sealing ring fits within the trough in a compression manner. While not shown, it should be understood that lubricant may be applied to the sealing ring 440 before or after being seated within the trough to facilitate sealing and rotational motion of the swivel segment 438 with respect to the sealing ring. Both flanges 450, 452 include generally smooth, circular outer walls 462 that are rounded over to transition into corresponding flat interior walls that delineate the trough, with the second flange having another flat wall that intersects a circular outer wall 466 of the conduit that is inset (includes a smaller outer diameter) with respect to the base 444. Proximate the end of the conduit 448 is a frustoconical flange 470 having a vertical wall 472 perpendicular to a conical wall 474 that terminates at the end. Reference will not be had to assembly of the aforementioned components.

Assembly includes seating the sealing rings 440 within the troughs of the swivel segments 438 so that each sealing ring is received within a different trough. Thereafter, the swivel segments 438 are oriented to so the flat walls at the bottom of each base 444 generally abut one another and the respective through holes 446 are longitudinally aligned with one another. In this orientation, the ends 449 of the conduits 448 of the swivel segments 438 point away (opposite) from one another. The bases 444 are likewise positioned to occupy the hollow interior of the sleeve 436. In exemplary form, the longitudinal length of the sleeve 436 is generally twice the longitudinal length of each base 444. Accordingly, when the bases 444 are oriented to abut one another and are located within the interior of the sleeve 436, the open ends of the sleeve are generally flush with the flat walls 464 of the second flange 452 of each base 444. This sub-assembled structure (sleeve and two swivel segments) is inserted between two housing 416 halves so that the interior of the housing halves is occupied by the sleeve and swivel segment bases 444.

In particular, the housing 416 halves are aligned so that the first set of ribs 426 operatively define corresponding cylindrical openings that are occupied by a conduit 448 of each swivel segment 438. At the same time, the housing 416 halves are aligned so that the second set of ribs 428 operatively define corresponding cylindrical openings that are occupied by the sleeve 436. As discussed above, the exterior surface of the sleeve 436 is adjacent to the innermost surface of the second set of ribs 428 and opposing open ends of the sleeve 436 abut complementary interior radial surfaces 442 of the first set of ribs 426 in order to wedge the sleeve between the first set of ribs. In this manner, the sleeve 436 does not appreciably move radially outward or longitudinally while seated within the housing 416 halves. Because of the longitudinal length of the conduits 448, the conduits extend through and beyond circular openings formed by the cooperation of the semicircular openings 424 of the frustums 422. As a result of the foregoing assembly, the swivel segments 438 are able to axially rotate with respect to one another, while maintaining a seal between the swivel segments. More specifically, one conduit 448 is mounted to the distal section 184 of the exhaust conduit, while the other conduit 448 is mounted to the proximal section 182 of the exhaust conduit. In this manner, the distal and proximal sections 182, 184 are able to rotate independent of one another.

The foregoing bases 444 and conduits 448 have been shown and described as being an integral structure. However, it should be realized that each swivel segment 438 may be fabricated from multiple pieces that are permanently mounted to one another or are removably mounted to one another.

Likewise, while the foregoing swivel section 412 provided a swivel for the exhaust conduit sections 182, 184, the same swivel structure may be duplicated and miniaturized to provide a swivel for the cryogenic feed conduit 128.

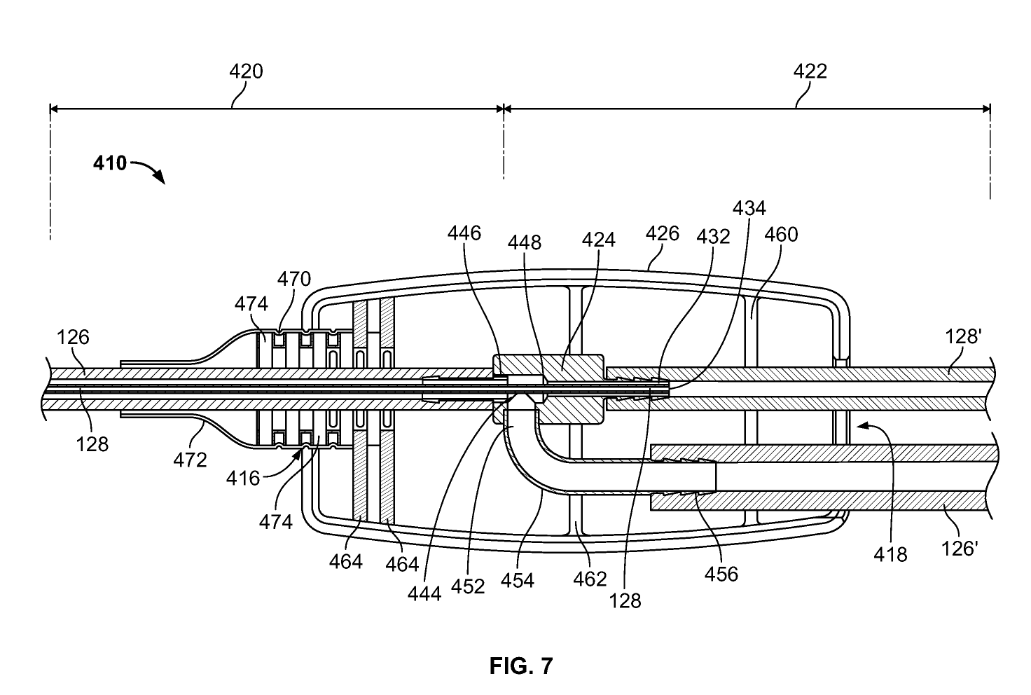

Referring to FIGS. 5 and 7, the diversion section 410 is upstream, with respect to the feed cryogenic fluid flow, from the swivel section 412. Similar to the diversion section 110 discussed in the first exemplary embodiment, this diversion section 410 operates to delineate between the coaxial section 420 and the parallel section 422 by using a diverter 424 within an insulating housing 426 comprised of complementary halves. It is to be understood, however, that the housing may be a single piece, such as an injection molded jacket. The housing 426 includes two openings 416, 418 oriented at opposite ends of the housing, with the larger of the two openings 418 located at a proximal end of the housing and allowing throughput of both the separate exhaust conduit 126′ and separate feed conduit 128′ from the diverter 424, whereas the smaller of the two openings 416 at the distal end of the housing allows throughput of the coaxial conduits 126, 128 to the diverter.

The diverter 424 includes a cryogenic feed barb fitting 432 that includes a smooth cylindrical cavity sized to receive the cryogenic feed conduit 128 and create a friction fit therewith. In this exemplary embodiment, the proximal end 434 of the barb fitting 432 marks the origination point of the cryogenic feed conduit 128, while the line 128′ in communication by the cryofluid reservoir 106 (see FIG. 5) extends distally past the proximal end to circumscribe the barb fitting 432.

The diverter 424 also includes a T-fitting 444 that operates to take the two coaxial fluid conduits (exhaust and cryogenic feed) and break these conduits up into two non-coaxial fluid conduits. The T-fitting 444 includes a first sub-fitting 446 that is coupled to the distal exhaust section 126. This sub-fitting 446 includes a longitudinal channel sufficient to accommodate the cryogenic fluid feed conduit 128 and provide a transition between the exhaust conduit 126 and the interior of the sub-fitting in order to carry the exhausted cryofluid further into the interior of the T-fitting 444. Within the interior of the T-fitting is a circumferential flange 448 having an orifice that is sized to accommodate only the diameter of the cryogenic feed conduit 128. More specifically, the exterior of the cryogenic feed conduit 128 and the portion of the flange 448 defining the orifice form a seal that prohibits passage of exhausted cryofluid from coaxially flowing over the top of the cryogenic feed conduit as the cryogenic feed conduit continues proximally within the diverter 424. Ultimately, the cryogenic feed conduit 128 terminates proximate the proximal end of barb fitting 432, which mounts to the cryogenic feed conduit 128′ that supplies cryogenic fluid from the supply reservoir 106.

Proximate the circumferential flange 448, the T-fitting 444 includes a perpendicular offshoot 452 in fluid communication with and circumscribed by a branch conduit 454 having its own barb fitting 456 at the proximal end thereof. This barb fitting 456 is similar to the barb fittings discussed above in terms of having inclined faces to provide a conical profile that allows the separate exhaust conduit 126′ to more easily circumscribe the fitting 456. At the same time, the barb fitting 456 includes vertical walls to retard proximal movement of the separate exhaust conduit 126′ after the separate exhaust conduit is installed around the fitting 456. In exemplary form, the branch conduit 454 is at least one of welded, brazed, and soldered to the perpendicular offshoot 452 to ensure a fluid tight seal therebetween.

The interior of the housing 416 includes a series of ribs 460, 462, 464 that protrude from an interior surface and operate to align the diverter 424 and conduits 126, 128, 126′, 128′. Distal from the diverter 424 is a strain adapter 470 that circumscribes the coaxial conduits 126, 128 and provides transition and support between the housing 426 and the coaxial conduits. The strain adapter 470 includes a polymeric cover 472 that tapers from distal to proximal and includes a series of circumferential ribs 474 that partially define a longitudinal channel occupied predominantly by the coaxial conduits 126, 128.

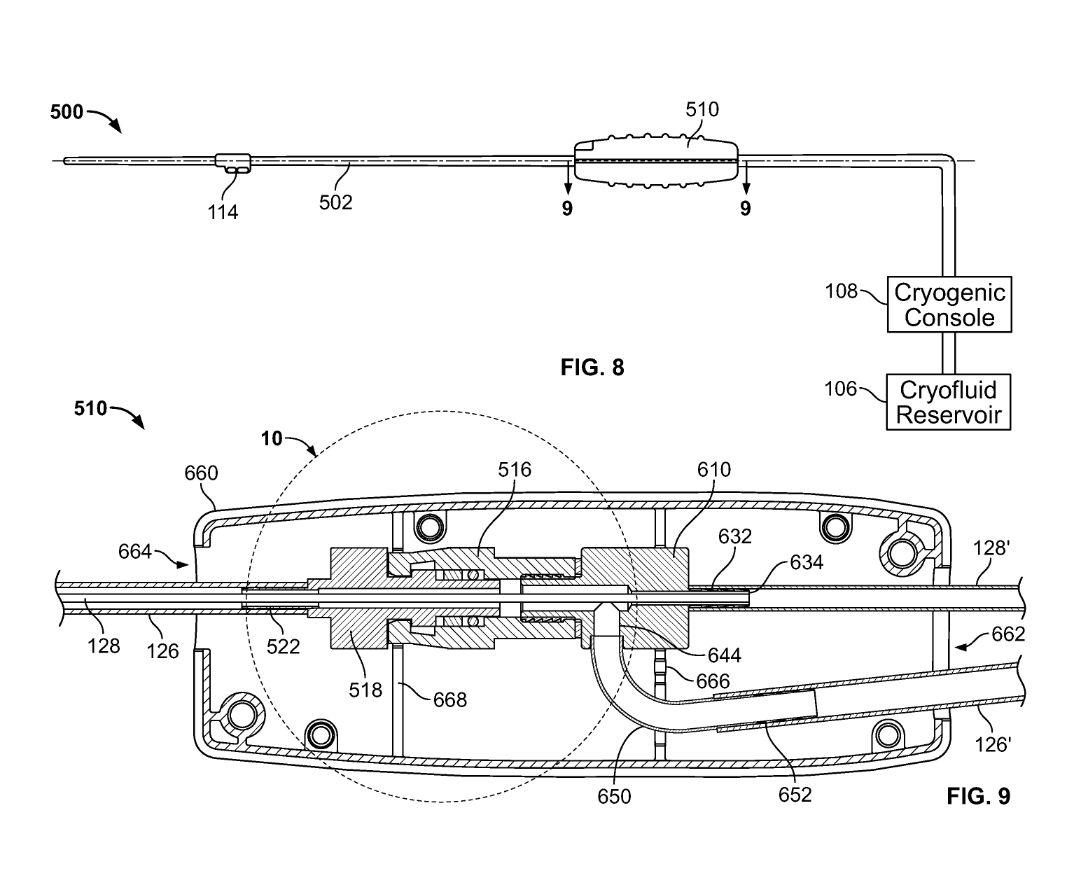

Referring to FIGS. 8-10, a third exemplary cryogenic probe 500 includes a wand 502 coupled to an umbilical tether 504 in order to supply cryogenic fluid from a fluid reservoir 106 to the wand. In this third exemplary embodiment, the flow of cryofluid to and from the cryogenic probe 500 is controlled from a separate, commercially available console 108 that regulates and controls the pressure of the cryofluid introduced into the cryogenic wand 502. The console 108 is capable of pressurizing the wand 502 for active defrost and provides for appropriate discharge of expanded cryofluid exhaust. Alternatively, or in addition, hand controls (not shown) may be associated with the cryogenic probe 500 to regulate and control the pressure of the cryofluid introduced into the cryogenic wand 502. In this third exemplary embodiment, the wand 502 includes an integrated swivel and diversion section 510 and a tool attachment section 114. Because this third exemplary probe 500 makes use of the same cryogenic fluid reservoir 106, console 108, and tool attachment section 114, a detailed description of these components will not be duplicated for purposes of brevity.

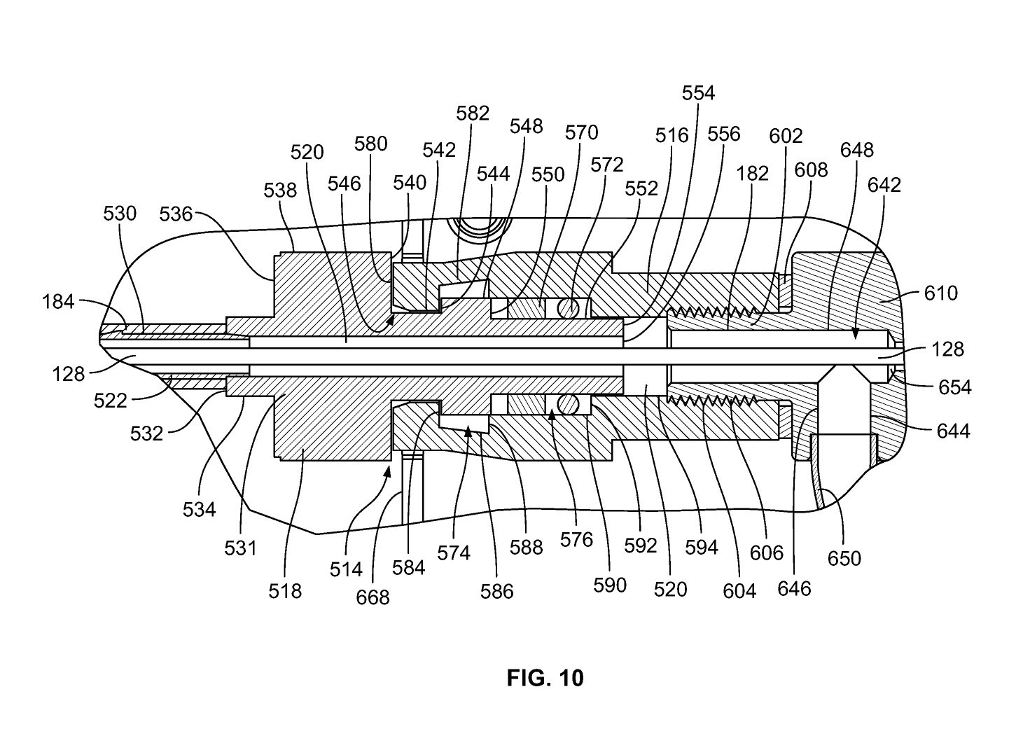

Referencing FIGS. 9 and 10, an exemplary integrated swivel and diversion section 510 includes a swivel diverter 512, which may be used in place of the diverters and swivels of the foregoing exemplary embodiments. The swivel diverter 512 includes a swivel 514 so that the proximal and distal sections 182, 184 of the exhaust conduit can rotate with respect to one another. In this exemplary embodiment, the swivel 514 includes a proximal component 516 and a distal component 518 that are rotationally repositionable with respect to one another. Each component 516, 518 includes a longitudinal passage 520 defined by a series of interior surfaces of the components that accommodates throughput of the feed conduit 128, but is also large enough to accommodate a separate stream of exhaust cryogenic fluid. More specifically, the interior circumferential surface of the feed conduit 128 provides a sealed delivery mechanism for the cryogenic fluid delivered from the cryogenic fluid tank. At the same time, the interior surfaces of the components 516, 518 cooperate with the exterior surface of the feed conduit 128 to define a sealed delivery mechanism for the exhausted cryogenic fluid.

The distal component 518 includes a barb fitting 522 having a circular opening that leads into a cylindrical cavity bounded by an interior surface having a substantially constant diameter. The circular opening is defined by a circular lip that transitions into the exterior surface 530 of the barb fitting 522. Beyond the barb, traveling proximally, the exterior surface is substantially cylindrical and is inset, within the primary distal component housing 531. This exterior of the distal component housing includes a first vertical wall 532 that intersects a first circumferential wall 534, which itself intersects a second vertical wall 536. This second vertical wall 536 intersects a second circumferential wall 538, which defines the maximum diameter of the distal component 518. Immediately proximal to this second circumferential wall 538 is a third vertical wall 540 that marks the transition where the exterior surfaces of the distal component 518 are contacted by and/or circumscribed by the proximal component 516. The third vertical wall 540 intersects a third circumferential wall 542, which itself intersects a fourth vertical wall 544. This fourth vertical wall 544 cooperates with the third circumferential wall 542 and the third vertical wall 540 to define a first circumferential groove 546 that extends around the exterior of the distal component 518. The fourth vertical wall 544 transitions into a fourth circumferential wall 548 that intersects a fifth vertical wall 550. The fifth vertical wall 550 intersects a fifth circumferential wall 552 that extends to the proximal tip 554 of the distal component 518. The proximal tip 554 includes a circular opening 556 that allows egress into the cylindrical longitudinal channel 520 that extends through the center of the distal component 518. This longitudinal channel 520 is occupied by a portion of the feed conduit 128, while the remaining area of the channel is available to act as the exhaust conduit 184.

As mentioned previously, a portion of the distal component 518 is circumscribed by a portion of the proximal component 516 and includes a first and second seal 570, 572 interposing the components to inhibit cryogenic exhaust from leaking between the components. Specifically, the first seal 570 is circular and has a rectangular cross-section and is spaced apart from the second seal 272, which is also circular, but has a circular cross-section. In this exemplary embodiment, the proximal and distal components 516, 518 cooperate to define a pair of circumferential cavities 574, 576, but only one of which is used to house the seals 570, 572. However, those skilled in the art will realize that one or more seals may be located in the first cavity 574. At the same time, those skilled in the art will realize that the second cavity 576 may have none, one, or more than one seal.

In order to create the cavities 574, 576, the proximal component 516 is hollow and includes a distal end 580 having a circumferential flange 582 that extends axially inward and is partially received within the first circumferential groove 546. A vertical, proximal wall 584 of the flange 582 contacts the fourth vertical wall 544 of the distal component 518 in order to retard the proximal component from proximally disengaging the distal component 518. Adjacent the proximal wall 584 is a conical wall 586 that intersects another vertical wall 588. The walls 584, 586, 588 of the proximal component 516 and the fourth circumferential wall 548 of the distal component generally define the first circumferential cavity 574. Adjacent to the vertical wall 588 is a cylindrical wall 590 that extends proximally until reaching a step 592, which intersects a second, smaller diameter cylindrical wall 594. The first cylindrical wall 588 and the step 592 of the proximal component 516 and the fifth circumferential wall 552 and fifth vertical wall 550 of the distal component 518 collectively generally define the second circumferential cavity 576 that houses the seals 570, 572. Those skilled in the art will realize that fewer or more than two seals 570, 572 may be utilized. Moreover, if multiple seals 570, 572 are utilized, any of various shapes of seals may be utilized. In addition, if multiple seals are utilized, the seals may all have the same cross-sectional shape and size, or the seals may have different shapes and/or sizes.

The swivel diverter 512 also includes a diverter 600 that is mounted to the proximal component 516 of the swivel 514. Specifically, the proximal end of the proximal component 516 is mounted to an adapter 602 of the diverter 600. In this exemplary embodiment, the proximal component 516 includes a proximal opening extending into its interior that is adapted to receive the adapter 602. Specifically, the interior of the proximal component 516 includes a threaded internal wall 604 that interfaces with threads 606 of the adapter 602 to secure the adapter 602 (overall, the diverter 600) to the proximal component. In order to ensure a fluid tight seal between the adapter 602 and the proximal component, a seal 608 interposes the proximal component and the housing 610 of the diverter 600.

The diverter 600 comprises a cryogenic feed barb fitting 632 that includes a smooth cylindrical cavity sized to receive the cryogenic feed conduit 128 and create a friction fit therewith. In this exemplary embodiment, the proximal end 634 of the barb fitting 632 marks the origination point of the cryogenic feed conduit 128, while the feed line 128′ in communication by the cryogenic fluid tank extends distally past the proximal end to circumscribe the barb fitting 632. The barb fitting 632 includes three barbs characterized by an inclined face (from proximal to distal) and a vertical wall intersecting the inclined face. The inclined faces provide a conical profile that allows the feed line to more easily circumscribe the fitting 632 and be repositioned distally, while the vertical walls retard proximal movement of the feed line after the feed line is installed around the fitting. The barb fitting 632 is an integral part of a diverter housing 610 having a longitudinal cavity through which the feed conduit 128 extends. The profile of the longitudinal cavity 642 extending through the diverter housing 610 changes from proximal to distal, with the distal portion being sized only to accommodate the cryogenic feed conduit 128, but moving distally, the profile increases to provide a portion of the exhaust conduit.

The diverter housing 610 includes a T-fitting 644 that operates to change the profile of the longitudinal cavity 642. The base section 646 of the T-fitting 644, which extends perpendicularly with respect an adapter section 648 and is in fluid communication therewith, is circumscribed by a branch conduit 650 having its own barb fitting 652 at the proximal end thereof. This barb fitting 652 is similar to the barb fitting 632 discussed above in terms of having inclined faces to provide a conical profile that allows the separate exhaust conduit 126′ to more easily circumscribe the fitting 652 and vertical walls to retard proximal movement of the separate exhaust conduit after the separate exhaust conduit is installed around the fitting 652. In exemplary form, the branch conduit 650 is at least one of welded, brazed, and, soldered, to the base section 646 to ensure a fluid tight seal therebetween.

In order to divert the exhaust cryogenic fluid through the branch conduit 650, the adapter section 648 is sealed at its proximal end 654 and is large enough to provide a circumferential gap between the interior of the branch conduit and the exterior of the feed conduit 128. This circumferential gap provides a pathway for exhaust cryogenic fluid passing from the exhaust conduit 126 to reach the base section 646, flow into the branch conduit 650, and ultimately flow into the separate exhaust conduit 126′.

The swivel diverter 512 is seated within a housing 660 comprised of complementary halves. It is to be understood, however, that the housing may be a single piece, such as an injection molded jacket. The housing 660 includes two openings 662, 664 oriented at opposite ends of the housing, with the larger of the two openings 662 located at a proximal end of the housing and allowing throughput of both the separate exhaust conduit 126′ and separate feed conduit 128′, whereas the smaller of the two openings 664 at the distal end of the housing allows throughput of the coaxial conduits 126, 128. The interior of the housing 660 includes a series of ribs 666, 668 that protrude from an interior surface and operate to align the diverter 124 and conduits 126, 128, 126′, 128′.

Following from the above description and disclosure summaries, it should be apparent to those of ordinary skill in the art that, while the methods and apparatuses herein described constitute exemplary embodiments of the present disclosure, the disclosure contained herein is not limited to this precise embodiment and that changes may be made to such embodiments without departing from the scope of the invention as defined by the claims. Additionally, it is to be understood that the invention is defined by the claims and it is not intended that any limitations or elements describing the exemplary embodiments set forth herein are to be incorporated into the interpretation of any claim element unless such limitation or element is explicitly stated. Likewise, it is to be understood that it is not necessary to meet any or all of the identified advantages or objects of the invention disclosed herein in order to fall within the scope of any claims, since the invention is defined by the claims and since inherent and/or unforeseen advantages of the present disclosure may exist even though they may not have been explicitly discussed herein.

What is claimed is:

1. A cryogenic wand for ablating tissue comprising:
a cryogenic ablation tube closed at a distal end and including an ablation zone, the ablation tube in fluid communication with and coupled proximally to at least one of two segments that are rotatably repositionable with respect to one another, wherein the two segments are fluidically sealed so that fluid entering a first of the at least two segments passes therethrough and enters a second of the at least two segments.

2. The cryogenic wand of claim 1, wherein a swivel interposes the at least two segments.

3. The cryogenic wand of claim 2, wherein the swivel circumscribes a stationary cryogenic feed conduit supplying fresh cryogenic fluid to the cryogenic ablation tube.

4. The cryogenic wand of claim 1, wherein the cryogenic ablation tube includes a thermocouple.

5. The cryogenic wand of claim 2, wherein the swivel comprises:
a first swivel section defining a first longitudinal cavity extending therethrough;
a second swivel section defining a second longitudinal cavity extending therethrough; and,
a first seal circumscribing at least a portion of the first swivel section and inscribing at least a portion of the second swivel section to form a seal between the first and second swivel sections.

6. The cryogenic wand of claim 5, wherein the first longitudinal cavity is in series with the second longitudinal cavity.

7. The cryogenic wand of claim 6, wherein:
an interior surface defining the first longitudinal cavity also defines a portion of a cryogenic fluid exhaust conduit;
an interior surface defining the second longitudinal cavity also defines a portion of the cryogenic fluid exhaust conduit; and,
the portions of the cryogenic; fluid exhaust conduit defined by the first and second longitudinal cavities circumscribe a portion of a cryogenic feed conduit.

8. The cryogenic wand of claim 5, wherein:
the first longitudinal cavity is coaxial with the second longitudinal cavity; and,
the first and second longitudinal cavities are at least partially occupied by a cryogenic feed conduit.

9. The cryogenic wand of claim 2, wherein the swivel comprises:
a first swivel section defining a first longitudinal cavity extending therethrough;
a second swivel section defining a second longitudinal cavity extending therethrough;
a sleeve concurrently circumscribing at least a portion of the first swivel section and a portion of the second swivel section; and,
a seal interposing the sleeve and at least one of the first swivel section and the second swivel section.

10. The cryogenic wand of claim 9, wherein:
the first swivel section includes a first circumferential recess;
the second swivel section includes a second circumferential recess;
the sleeve is cylindrical and concurrently circumscribes the first circumferential recess and the second circumferential recess; and,
the seal includes a first O-ring seated within the first circumferential recess to interpose the sleeve and the first swivel section to seal therebetween, and the seal includes a second O-ring seated within the second circumferential recess to interpose the sleeve and the first swivel section to seal therebetween.

11. The cryogenic wand of claim 10, wherein:
the first swivel section includes a first cylindrical portion;
the second swivel section includes a second cylindrical portion;
the sleeve houses both the first and second cylindrical portions; and,
an insulated housing circumscribes the sleeve, the first cylindrical portion, and the second cylindrical portion.

12. The cryogenic wand of claim 1, further comprising:
a diverter in fluid communication with the cryogenic ablation tube, the diverter including:
a first fitting accommodating an outgoing cryogenic feed conduit and an incoming cryogenic exhaust conduit,
a second fitting accommodating an incoming cryogenic feed conduit, and
a third fitting accommodating an outgoing cryogenic-exhaust conduit,
wherein the second fitting is sealed off from the third fitting.

13. The cryogenic wand of claim 12, wherein the first fitting is operatively coupled to at least one of the at least two segments that are rotatably repositionable with respect to one another.

14. The cryogenic wand of claim 12, wherein the diverter is positioned within an insulated housing, where the insulating housing includes a first orifice receiving at least two coaxial conduits, where a first of the at least two coaxial conduits is an outgoing cryogenic feed conduit, and where a second of the at least two coaxial conduits is incoming cryogenic exhaust conduit.

15. The cryogenic wand of claim 14, wherein the insulating housing includes a second orifice receiving at least one of an incoming cryogenic feed conduit and an outgoing cryogenic exhaust conduit.

16. The cryogenic wand of claim 15, wherein:
an incoming cryogenic feed conduit enters the insulating housing and an outgoing cryogenic exhaust conduit leaves the insulating housing; and,
the incoming cryogenic feed conduit and the outgoing cryogenic exhaust conduit are not coaxially oriented with respect to one another.

17. The cryogenic wand of claim 1, further comprising a robotic appendage coupled to the cryogenic ablation tube in order to facilitate grasping by robotic jaws.

18. The cryogenic wand of claim 17, wherein the robotic appendage includes a collar that circumscribes cryogenic ablation tube.

19. The cryogenic wand of claim 18, wherein the collar defines a longitudinal cavity occupied by at least a portion of the cryogenic ablation tube and an adapter to couple the cryogenic tube to at least one of the at least two segments that are rotatably repositionable with respect to one another.

20. The cryogenic wand of claim 1, further comprising an integrated diverter and swivel assembly that interposes the at least two segments that are rotatably repositionable with respect to one another, wherein a first of the at least two segments includes at least two separate conduits for carrying cryogenic feed fluid and exhausted cryogenic fluid, wherein a second of the at least two segments includes at least two separate conduits for carrying the cryogenic feed fluid and the exhausted cryogenic fluid.

21. The cryogenic wand of claim 20, wherein:
the first of the at least two segments comprises a first swivel section;
the second of the at least two segments comprises a second swivel section; and,
the integrated diverter and swivel assembly includes a first seal circumscribing at least a portion of the first swivel section and inscribing at least a portion of the second swivel section to form a seal between the first and second swivel sections.

22. The cryogenic wand of claim 21, wherein the at least two separate conduits for carrying cryogenic feed fluid and exhausted cryogenic fluid of the first segment are coaxially oriented with respect to one another.

23. The cryogenic wand of claim 22, wherein the at least two separate conduits for carrying cryogenic feed fluid and exhausted cryogenic fluid of the second segment are not coaxially oriented with respect to one another.

24. The cryogenic wand of claim 20, wherein:
the first of the at least two segments comprises a first swivel section;
the second of the at least two segments comprises a second swivel section;
the integrated diverter and swivel assembly includes a sleeve concurrently circumscribing at least a portion of the first swivel section and a portion of the second swivel section; and,
the integrated diverter and swivel assembly includes a seal interposing the sleeve and at least one of the first swivel section and the second swivel section.

25. The cryogenic wand of claim 24, wherein the at least two separate conduits for carrying cryogenic feed fluid and exhausted cryogenic fluid of the first segment are coaxially oriented with respect to one another.

26. The cryogenic wand of claim 25, wherein the at least two separate conduits for carrying cryogenic feed fluid and exhausted cryogenic fluid of the second segment are not coaxially oriented with respect to one another.

27. The cryogenic wand of claim 24, wherein:
the first swivel section includes a first circumferential recess;
the second swivel section includes a second circumferential recess;
the sleeve is cylindrical and concurrently circumscribes the first circumferential recess and the second circumferential recess; and,
the seal includes a first O-ring seated within the first circumferential recess to interpose the sleeve and the first swivel section to seal therebetween, and the seal includes a second O-ring seated within the second circumferential recess to interpose the sleeve and the first swivel section to seal therebetween.

28. The cryogenic wand of claim 27, wherein:
the first swivel section includes a first cylindrical portion;
the second swivel section includes a second cylindrical portion;
the sleeve houses both the first and second cylindrical portions; and,
an insulated housing circumscribes the sleeve, the first cylindrical portion, and the second cylindrical portion.

29. A cryogenic probe for ablating tissue comprising:
an elongated tube open at a proximal end and closed at a distal end, the elongated tube operatively coupled to at least two segments that are rotatably repositionable with respect to one another using an in-series swivel, wherein the two segments are sealed to inhibit fluid passing between the at least two segments, the elongated tube defining an internal cavity; and,
at least one cryogenic fluid supply line occupying at least a portion of the internal cavity of the elongated tube, the at least one cryogenic fluid supply line including a nozzle for introducing cryogenic fluid into the internal cavity of the elongated shell.

30. The cryogenic probe of claim 29, wherein the at least one cryogenic fluid supply line includes at least two sections that are rotatably repositionable with respect to one another.

31. The cryogenic probe of claim 29, wherein the elongated tube includes a wall thickness of from about 0.020 inches to 0.035 inches.

32. The cryogenic probe of claim 29, wherein the elongated tube includes an outside diameter of from about 0.16 inches to about 0.20 inches.

33. The cryogenic probe of claim 29, further comprising a swivel interposing the at least two segments.

34. The cryogenic probe of claim 29, further comprising:
a diverter in fluid communication with the elongated tube, the diverter including:
a first fitting accommodating an outgoing cryogenic feed conduit and an incoming cryogenic exhaust conduit,
a second fitting accommodating an incoming cryogenic feed conduit, and
a third fitting accommodating an outgoing cryogenic exhaust conduit,
wherein the second fitting is sealed off from the third fitting.

35. The cryogenic probe of claim 29, further comprising a thermocouple mounted to the elongated tube.

35. The cryogenic probe of claim 29, further comprising a metallic coil housed within the elongated tube.

36. A cryogenic probe for ablating tissue comprising:
an ablator comprising an ablation tube closed at one end and occupied by a cryogenic feed tube having an orifice, where an interior surface of the ablator at least partially defines a cryogenic exhaust conduit, where the ablation tube includes a wall thickness of from about 0.020 inches to 0.035 inches, and where the ablation tube includes an outside diameter of from about 0.16 inches to about 0.20 inches; and,
a swivel operatively coupled to the ablator to allow the ablation tube to rotate about the cryogenic feed tube.

37. A method of fabricating a cryogenic wand for ablating tissue, the method comprising:
forming a cryogenic ablation tube closed at one end; and,
operatively coupling the cryogenic ablation tube to a swivel, where the swivel interposes the closed end of the cryogenic ablation tube and a conduit adapted to carry at least one of feed cryogenic fluid proximate the closed end of the cryogenic ablation tube and exhaust cryogenic fluid returning from proximate the closed end of the cryogenic ablation tube.

38. The method of claim 37, further comprising the act of inserting a cryogenic feed conduit within the swivel so the swivel circumscribes the cryogenic feed conduit.

39. The method of claim 37, further comprising the act of inserting mounting a thermocouple to the cryogenic ablation tube.

40. The method of claim 37, wherein the swivel comprises:
a first swivel section defining a first longitudinal cavity extending therethrough;
a second swivel section defining a second longitudinal cavity extending therethrough; and,
a first seal circumscribing at least a portion of the first swivel section and inscribing at least a portion of the second swivel section to form a seal between the first and second swivel sections.

41. The method of claim 40, wherein the first longitudinal cavity is in series with the second longitudinal cavity.

42. The method of claim 41, wherein:
an interior surface defining the first longitudinal cavity also defines a portion of a cryogenic fluid exhaust conduit;
an interior surface defining the second longitudinal cavity also defines a portion of the cryogenic fluid exhaust conduit; and,
the portions of the cryogenic fluid exhaust conduit defined by the first and second longitudinal cavities circumscribe a portion of a cryogenic feed conduit.

43. The method of claim 41, wherein:
the first longitudinal cavity is coaxial with the second longitudinal cavity; and
the first and second longitudinal cavities are at least partially occupied by a cryogenic feed conduit.

44. The method of claim 37, wherein the swivel comprises:
a first swivel section defining a first longitudinal cavity extending therethrough;
a second swivel section defining a second longitudinal cavity extending therethrough;
a sleeve concurrently circumscribing at least a portion of the first swivel section and a portion of the second swivel section; and,
a seal interposing the sleeve and at least one of the first swivel section and the second swivel section.

45. The method of claim 44, wherein:
the first swivel section includes a first circumferential recess;
the second swivel section includes a second circumferential recess;
the sleeve is cylindrical and concurrently circumscribes the first circumferential recess and the second circumferential recess; and,
the seal includes a first O-ring seated within the first circumferential recess to interpose the sleeve and the first swivel section to seal therebetween, and the seal includes a second O-ring seated within the second circumferential recess to interpose the sleeve and the first swivel section to seal therebetween.

46. The method of claim 45, wherein:
the first swivel section includes a first cylindrical portion;
the second swivel section includes a second cylindrical portion;
the sleeve houses both the first and second cylindrical portions; and,
an insulated housing circumscribes the sleeve, the first cylindrical portion, and the second cylindrical portion.

47. The method of claim 37, further comprising the act of operatively coupling a diverter to the swivel, the diverter including:
a first fitting accommodating an outgoing cryogenic feed conduit and an incoming cryogenic exhaust conduit,
a second fitting accommodating an incoming cryogenic feed conduit, and
a third fitting accommodating an outgoing cryogenic exhaust conduit,
wherein the second fitting is sealed off from the third fitting.

48. The method of claim 47, wherein the first fitting is operatively coupled to at least one of the at least two segments that are rotatably repositionable with respect to one another.

49. The method of claim 47, wherein the diverter is positioned within an insulated housing, where the insulating housing includes a first orifice receiving at least two coaxial conduits, where a first of the at least two coaxial conduits is an outgoing cryogenic feed conduit, and where a second of the at least two coaxial conduits is incoming cryogenic exhaust conduit.

50. The method of claim 49, wherein the insulating housing includes a second orifice receiving at least one of an incoming cryogenic feed conduit and an outgoing cryogenic exhaust conduit.

51. The method of claim 50, wherein:
an incoming cryogenic feed conduit enters the insulating housing and an outgoing cryogenic exhaust conduit leaves the insulating housing; and,
the incoming cryogenic feed conduit and the outgoing cryogenic exhaust conduit are not coaxially oriented with respect to one another.

52. The method of claim 37, further comprising the act of mounting a robotic appendage to the cryogenic ablation tube in order to facilitate grasping by robotic jaws.

53. The method of claim 52, wherein the robotic appendage includes a collar that circumscribes cryogenic ablation tube.

54. The method of claim 53, wherein the collar defines a longitudinal cavity occupied by at least a portion of the cryogenic ablation tube and an adapter to couple the cryogenic tube to at least one of the at least two segments that are rotatably repositionable with respect to one another.

55. The method of claim 37, wherein the swivel comprises an integrated diverter and swivel assembly, wherein the integrated diverter and swivel assembly is operative to take an input of two coaxial conduits and create an output of two non-coaxial conduits.

56. The cryogenic wand of claim 1, wherein the cryogenic ablation tube includes a wall that is corrugated.

57. The cryogenic probe of claim 29, wherein the elongated tube includes a wall that is corrugated.

58. A cryogenic probe for ablating tissue comprising:
an elongated tube open at a proximal end and closed at a distal end, the elongated tube operatively coupled to a conduit rotatably repositionable with respect to the elongated tube using an in-series swivel, wherein the swivel is sealed to inhibit fluid passing between swivel joints, the elongated tube defining an internal cavity; and,
at least one cryogenic fluid supply line occupying at least a portion of the internal cavity of the elongated tube, the at least one cryogenic fluid supply line including a nozzle for introducing cryogenic fluid into the internal cavity of the elongated shell.
